(19) United States
(12) Patent Application Publication
(10) Pub. No.: US 2024/0376125 A1
(43) Pub. Date: Nov. 14, 2024

(54) MECHANOPHORE WITH FORCE TRIGGERED STEREOCHEMISTRY CONVERSION

(71) Applicant: Xiaoran Hu, Syracuse, NY (US)

(72) Inventor: Xiaoran Hu, Syracuse, NY (US)

(73) Assignee: Syracuse University, Syracuse, NY (US)

(21) Appl. No.: 18/656,114

(22) Filed: May 6, 2024

Related U.S. Application Data
(60) Provisional application No. 63/464,213, filed on May 5, 2023.

(51) Int. Cl. C07D 513/04

(52) U.S. Cl. CPC C07D 513/04; C07B 2200/07

(57) ABSTRACT

A parallel diarylethene mechanophore with mirror symmetry may be converted to its anti-parallel stereoisomer of C2 symmetry under ultrasound-induced force field. Concomitantly, the stereochemistry-converted material gains new stereochemistry-selective activity to undergo photocyclization reactions. The mechanophore thus uses a mechanical approach to converting the axial chirality of a sterically congested diarylethene molecule, modifying its molecular symmetry and its physical and chemical properties in response to an mechanical stimulus.

### CROSS-REFERENCE TO RELATED APPLICATIONS

The present application claims priority to U.S. Provisional App. No. 63/464,213, filed on May 5, 2023.

### BACKGROUND

### 1. Field

The present disclosure relates to mechanophores and, more specifically, to a molecule that will undergo a chirality change in response to an external force.

### 2. Description of the Related Art

Stereochemistry influences the physical properties and chemical reactivities of materials through the three-dimensional arrangement of atoms within molecules. Rational manipulation of stereochemistry, such as the E-Z geometric isomerism, using external stimuli presents a potent strategy for regulating the physical, chemical and biological properties of materials. Previously, the conversion of material stereochemistry has been achieved through the application of stimuli such as light and chemical reagents. Mechanical stress is ubiquitous in biological organisms and synthetic materials, but few have investigated the interaction between molecular stereochemistry and mechanical force. Atropisomerism is a type of axial chirality that is ubiquitously found in the field of medicinal chemistry, catalysis, and materials science. Atropisomers are stereoisomers with axial chirality arising because of hindered rotation about a single bond. Diarylethenes (DAEσ) are an intriguing class of photoswitchable molecules that exist in two atropisomer states that usually undergo rapid atropisomerization, namely the parallel and anti-parallel conformers (Scheme 1). The anti-parallel DAE conformer has C2 rotational symmetry and undergoes reversible disrotatory 6π photocyclization between the ring-open colorless state and ring-closed colored state, while its thermal electrocyclic reaction in the ground state is symmetry-forbidden according to Woodward-Hoffman rules. In comparison, the parallel conformer with mirror symmetry does not cyclize in neither thermal nor photochemical conditions because of its unfavorable steric interaction and molecular symmetry. Interestingly, other groups have found that steric congestion between the ethene bridge and the side-arm aryl substituents in DAEσ hinders the rotation around the ethene-aryl single bonds. The two ring-open atropisomers of those crowded DAEσ are locked in their respective conformations under ambient conditions, and individual atropisomers have been successfully isolated and their properties studied.

The last two decades of research in polymer mechanochemistry has provided a collection of stress-responsive molecules known as mechanophores with different functional responses to mechanical stimulation such as color change, activation of catalysis, switching of electrical conductivity, and generation of reactive species. The fundamental mechanisms behind reported force-matter interactions are summarized into two general categories: covalent and non-covalent transformations. Most covalent mechanochemical transformations fall under homolytic cleavage, heterolytic cleavage, pericyclic, or metal-coordinate bond cleavage. On the other hand, the development of mechanophores harnessing non-covalent molecular transformation is an emerging research topic. For example, some researchers have developed a series of rotaxane- and cyclophane-based supramolecular mechanophores, where mechanical force affects the spatial alignment between chromo-phores and alters their photoluminescent properties. Saito and coworkers designed “flapping” mechanophores that undergo conformational planarization under force stimulation which extends the conjugation length. Similarly, other groups have developed mechanophores based on twisted conjugated systems that planarize and gain conjugation efficiency under force. Additionally, Herrmann, Gostl, and coworkers have explored strategies to control host-guest interactions with force in protein and aptamer materials. This document describes a type of advanced mechanoresponsive materials with stereochemistry-dependent properties for use in stress-sensing, medicinal chemistry, information storage, lithography, and chiroptic applications.

### BRIEF SUMMARY

The present invention provides a mechanical approach to converting the stereochemistry of a molecule, thereby modifying its physical and chemical properties. For example, the atropisomer stereochemistry of a sterically congested diarylethene molecule can be converted by the application of mechanical force, thereby modifying its physical and chemical properties. The parallel diarylethene mechanophore with mirror symmetry is converted to its anti-parallel stereoisomer of C₂ symmetry under ultrasound-induced force field. Concomitantly, the chirality-converted material changes its fluorescence emission and gains new chirality-selective activity to undergo photocyclization reactions. Regulating material properties through force-stereochemistry coupling serves as a general design strategy for configurational mechanophores and may lead to the development of advanced mechanoresponsive materials with stereochemistry-dependent physicochemical and biological properties that are potentially useful in stress-sensing, medicinal chemistry, information storage, lithography, and chiroptic applications

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic an exemplary of parallel DAEσ mechanophore according to the present invention that illustrates how a mechanical force can be used to modify atropisomer stereochemistry, molecular symmetry, and chemical activity mechanophores.

FIG. 2 is a schematic of a design of a generic structure for a mechanophore according to the present invention.

FIG. 3 is a schematic of a series of additional structures for a mechanophore according to the present invention.

FIG. 4 is a graph of a DFT calculation using the CoGEF technique predicts the force-triggered conversion of the parallel DAEσ to its antiparallel stereoisomer DAE-C₂.

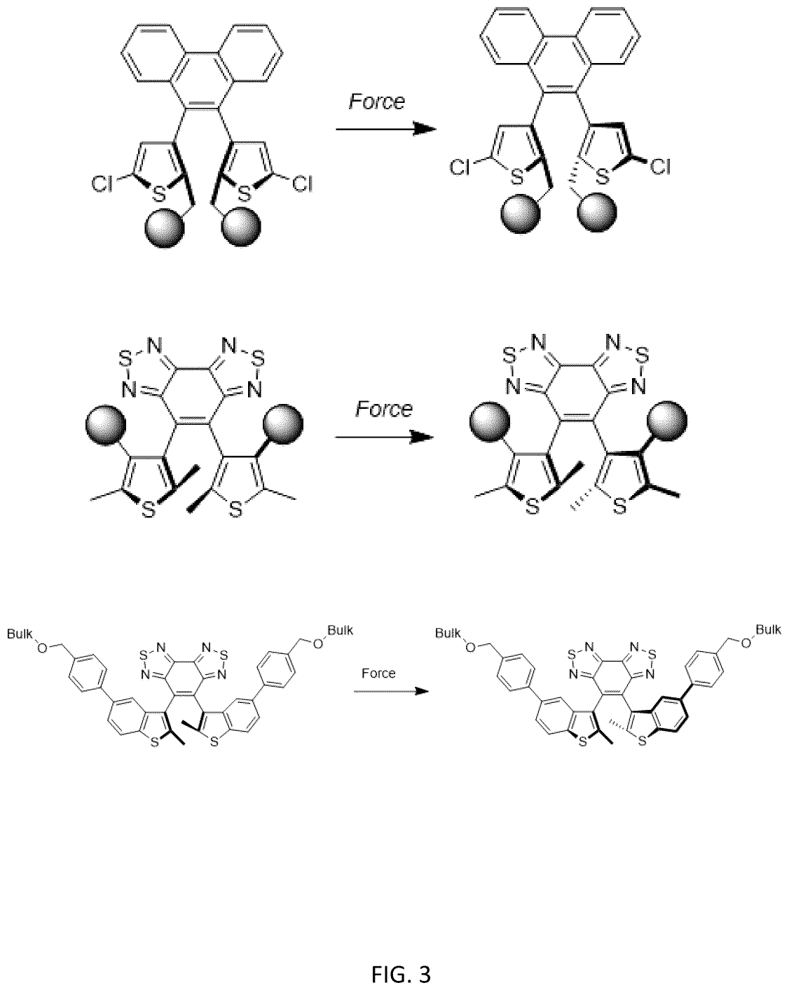

FIG. 3 is a scheme illustrating the synthesis of Polymer P1σ containing a chain-centered parallel DAE mechanophore with mirror symmetry.

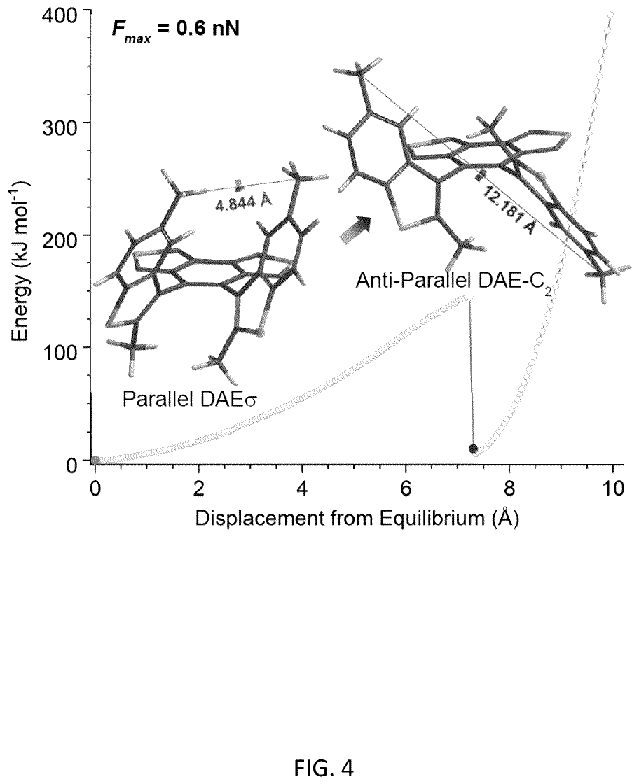

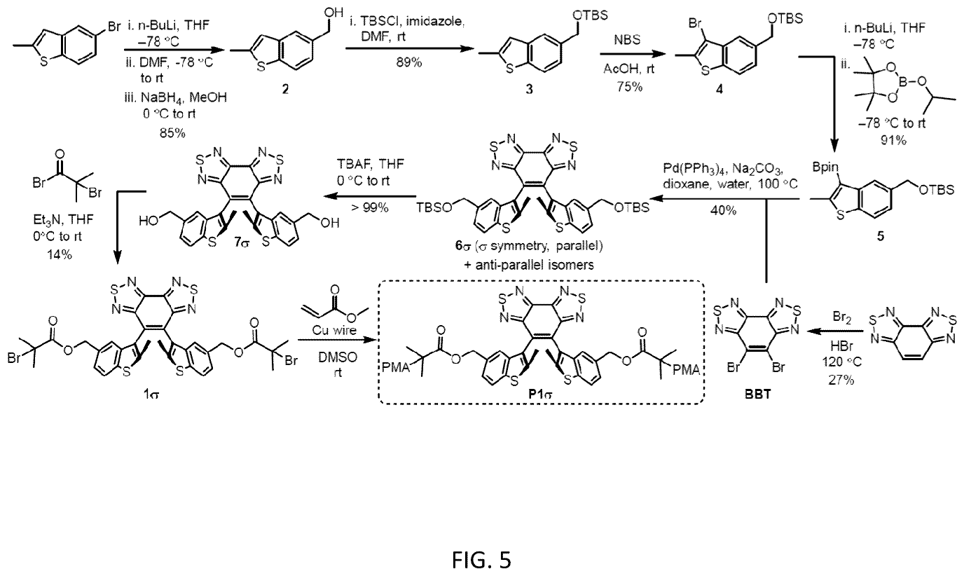

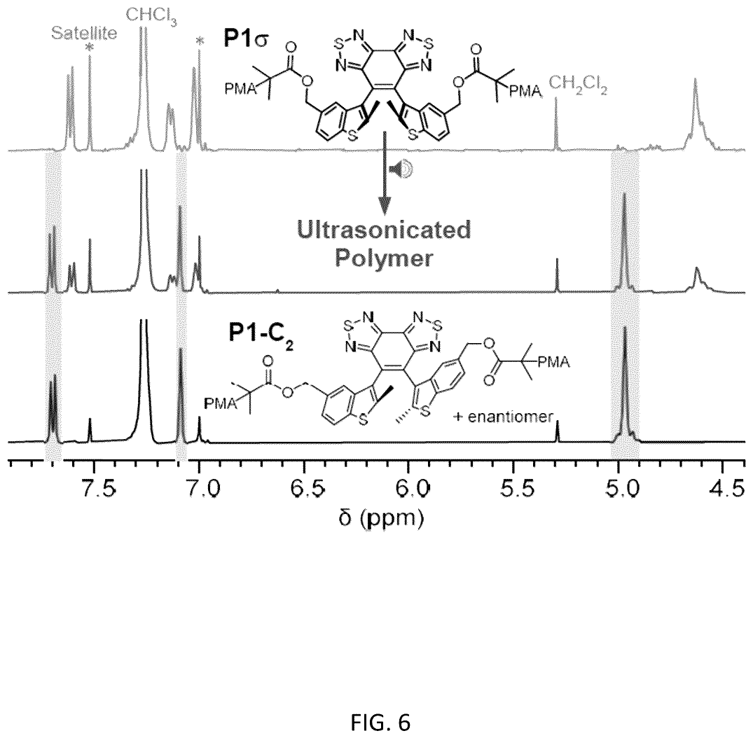

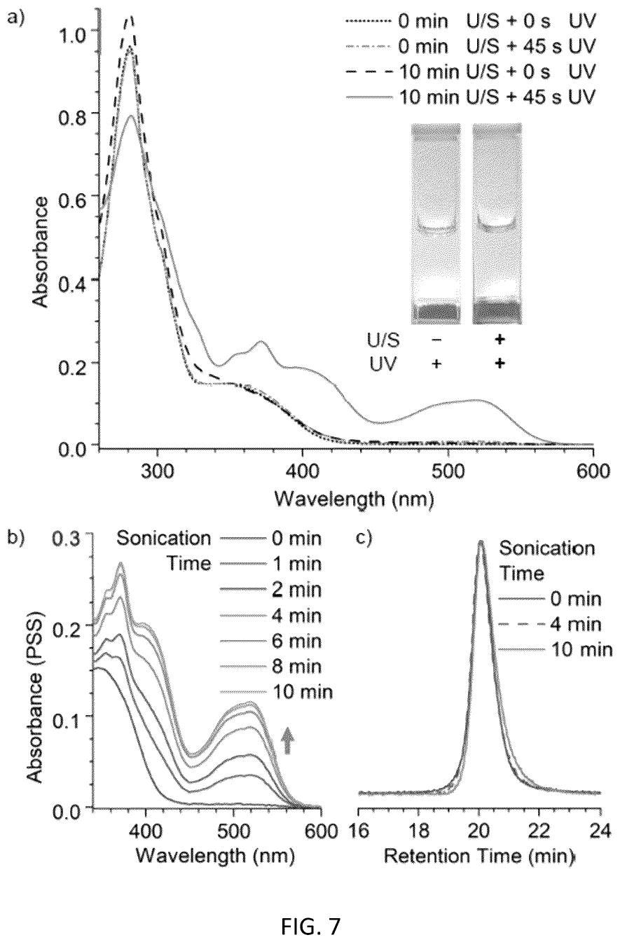

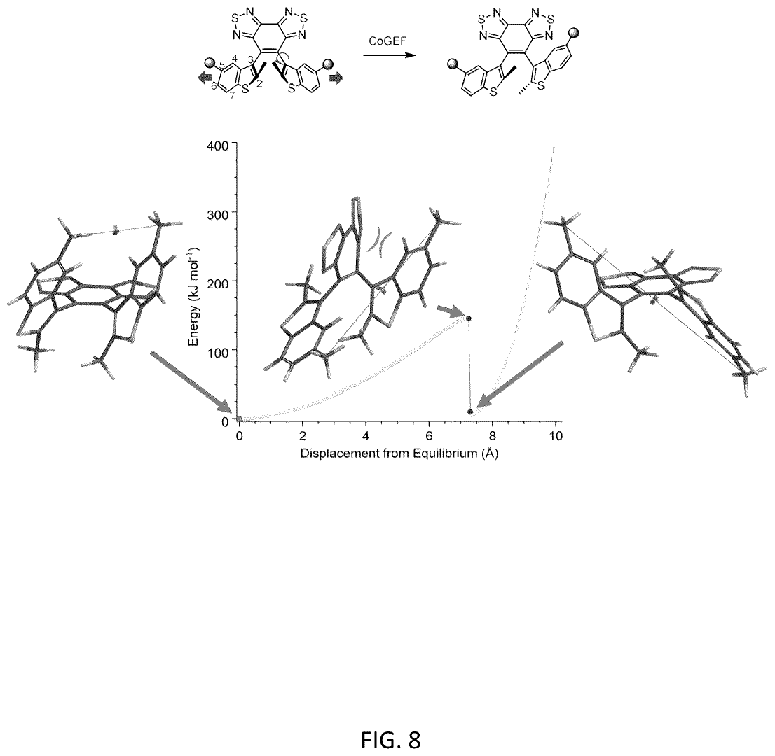

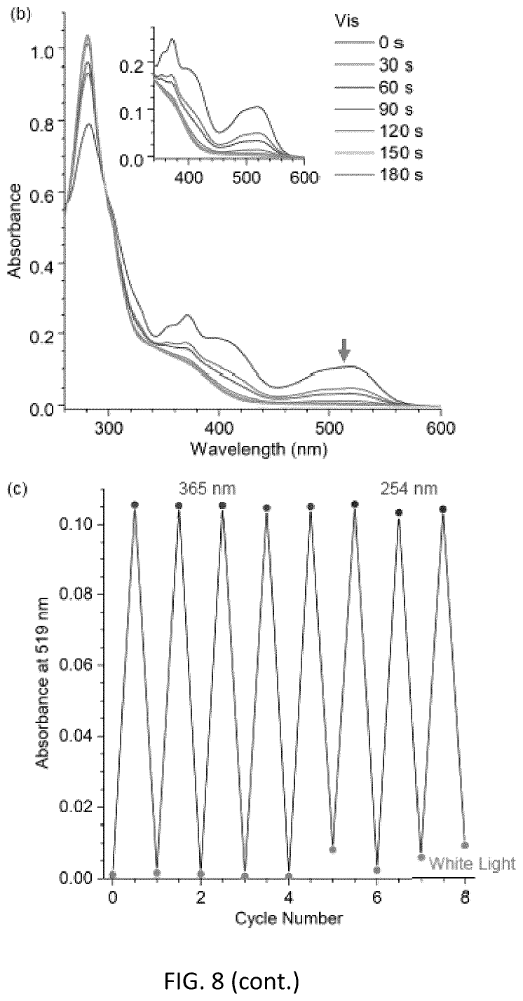

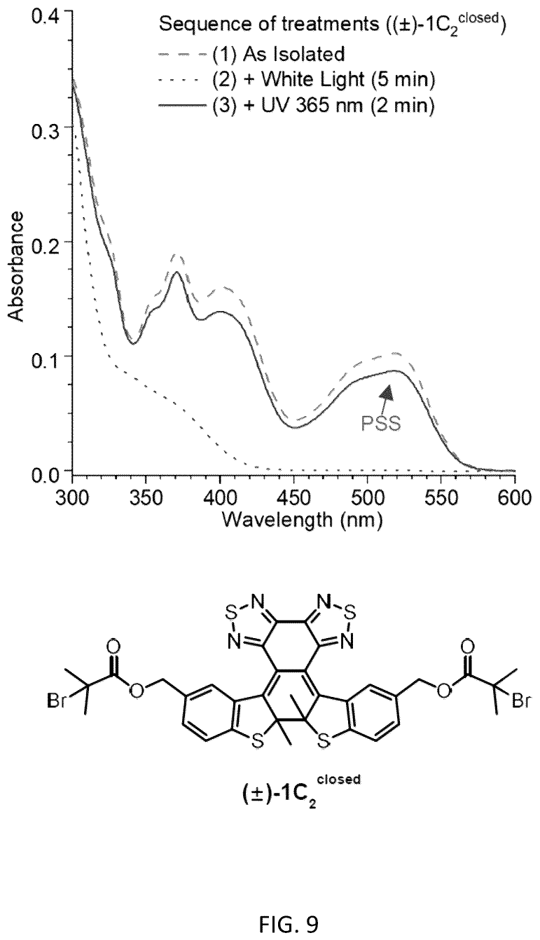

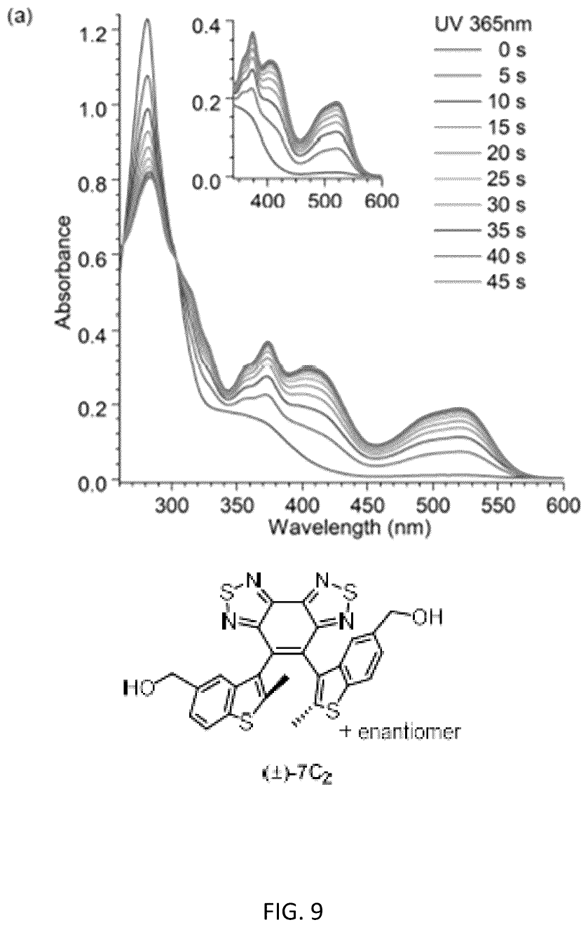

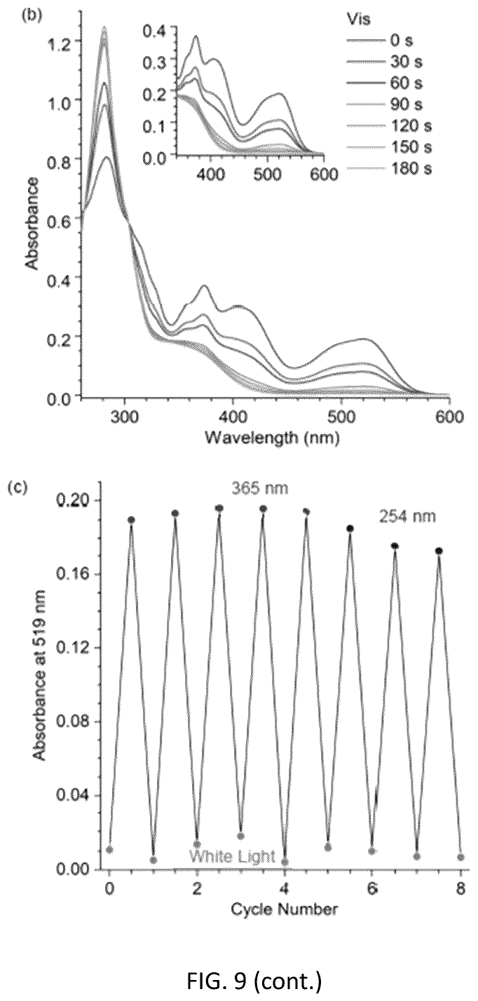

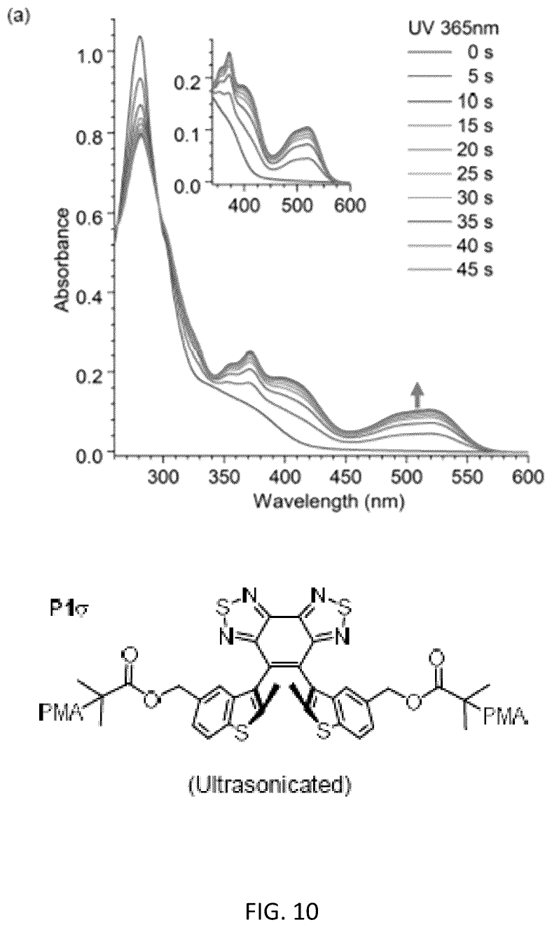

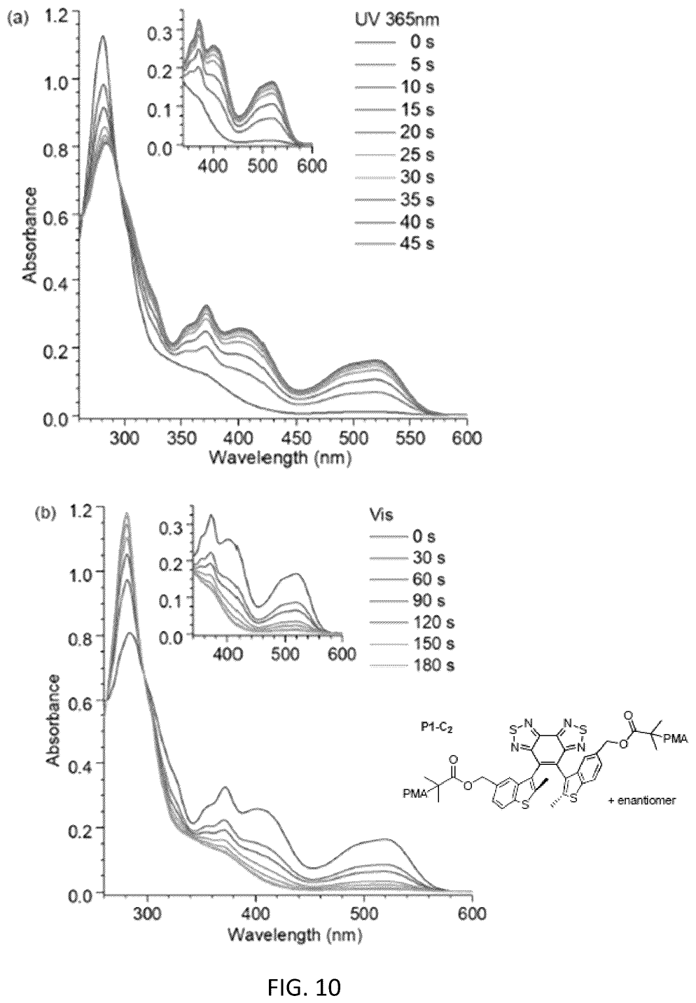

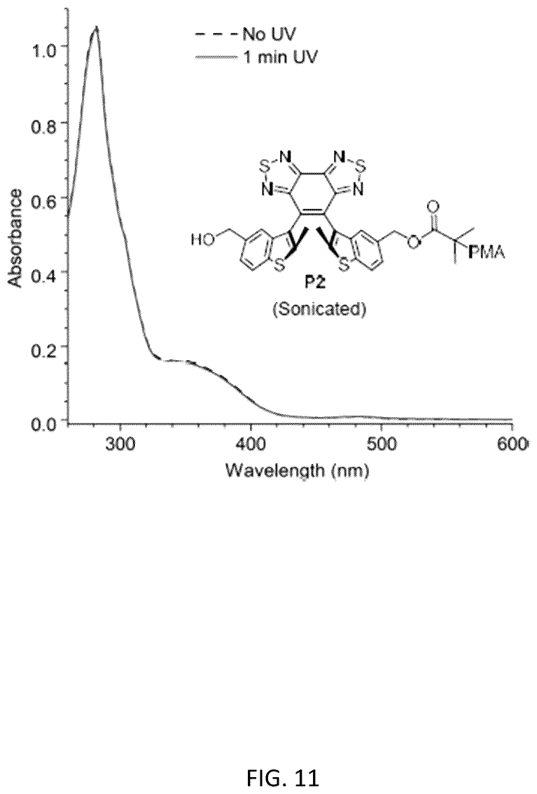

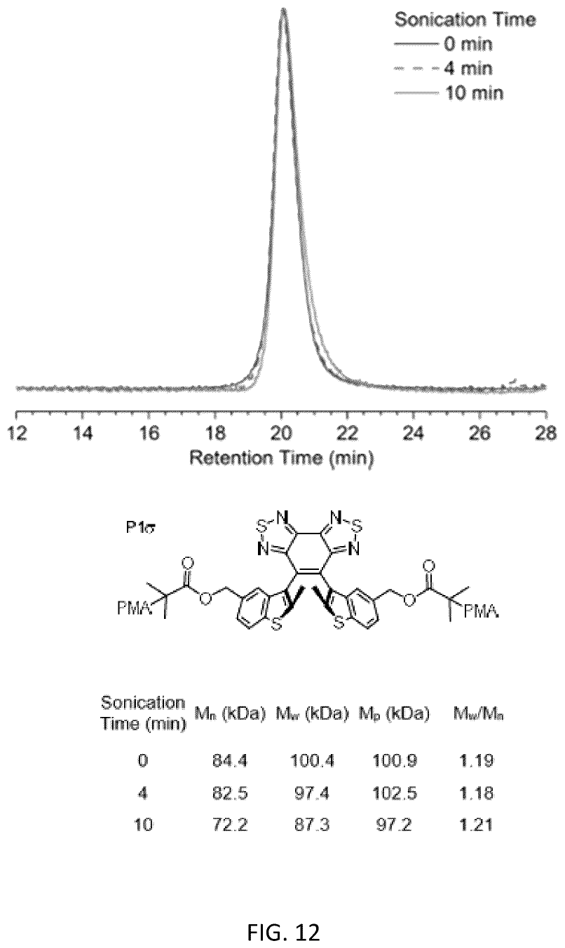

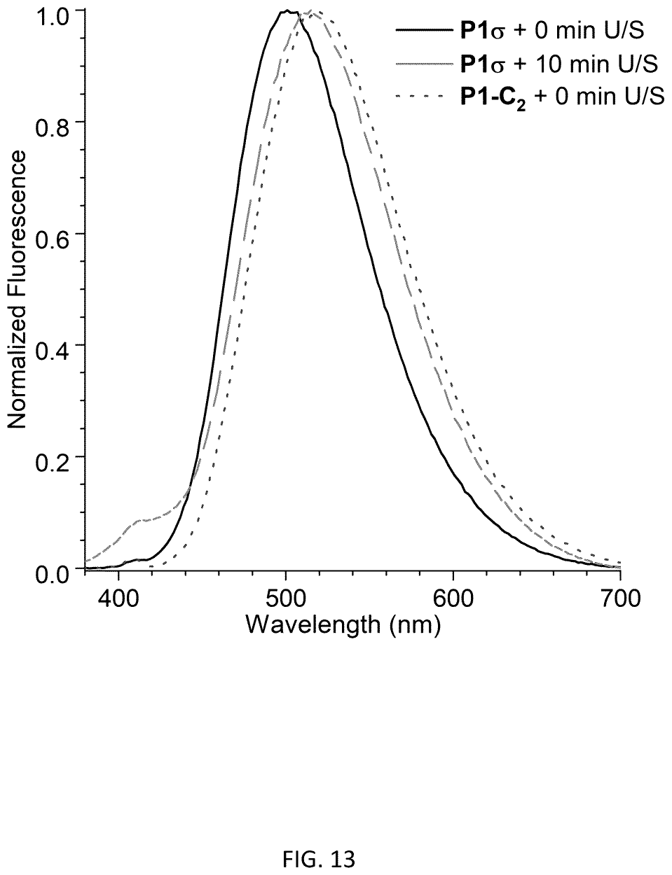

FIG. 4 is a graph of the 1H NMR spectra (400 MHZ, CDCl3) of P1σ before (top) and after (middle) ultrasonication provide direct evidence for the U/S-triggered conversion of the parallel DAEσ to its anti-parallel stereoisomer DAE-C2. An acetonitrile solution of P1σ (36 mL, 2.0 mg/mL) was subjected to standard ultrasonication conditions, concentrated, and precipitated into cold methanol to afford the ultrasonicated polymer sample used in the NMR analysis.

FIG. 5 is (a) Ultrasonication-dependent photochromism of P1σ demonstrates that force triggers the conversion of the photoinert parallel DAEσ mechanophore into its anti-parallel stereoisomer DAE-C2. The resulting material gains stereoselective activity to undergo conrotatory photochemical electrocyclic reaction between the ring-open colorless (black dashed line) and the ring-closed colored forms (red solid line). (b) Absorption spectra of sonicated P1σ solutions in PSS as a function of ultrasonication time indicate the stereochemistry conversion plateaued after ˜10 min ultrasonication. PSS was achieved by 45 s irradiation (365 nm) under a handheld UV lamp. (c) Normalized GPC traces of P1σ as a function of ultrasonication time. Pulsed U/S (1 s on/2 s off) was applied to an acetonitrile solution of P1σ (12 mL, 2.0 mg/mL) at 0° C.

FIG. 6 is a graph of the structure of the truncated 5-substituted DAE at equilibrium geometry, immediately prior to the stereochemistry conversion, and immediately after the mechanical conversion. The maximum force 0.6 nN was calculated from the slope of the curve.

FIG. 7 is a graph of the UV-vis absorption spectra of a dilute acetonitrile solution of (±)-1C₂^{closed} after a sequence of manipulations—Red dashed line: as isolated (±)-1C₂^{closed} dissolved in acetonitrile; Black dotted line: after 5 min visible light irradiation; Blue solid line (PSS): after 2 min UV irradiation at 365 nm. The percentage conversion in the PSS was calculated: DAEclosed %=OD_{PSS}/ODclosed=85%.

FIG. 8 is a series of graphs of the chemical structure of polymer P1σ and its photochemical property after 10 min ultrasonication (2 mg/mL in acetonitrile) characterized by UV-vis spectroscopy. (a) Photochromism of the P1σ solution under 365 nm UV irradiation. The PSS was achieved after about 40 s irradiation. (b) Subsequent visible-light irradiation of this polymer solution resulted in the ring-opening reaction and discoloration. The color completely disappeared after about 150 s visible irradiation using a white flashlight. (c) Reversible photochromism of the sonicated P1σ solution under UV (λ=365 nm or 254 nm) and visible light. The absorbance was monitored at the absorption peak of the ring-closed DAE at 519 nm.

FIG. 9 is a series of graphs of the chemical structure of a model photoswitch molecule (±)-7C₂ and its photochemical property in acetonitrile characterized by UV-vis spectroscopy. (a) Photochromism of the (±)-7C₂ solution under 365 nm UV irradiation. The PSS was achieved after about 40 s irradiation. (b) Subsequent visible-light irradiation of this (±)-7C₂ solution resulted in the ring-opening reaction and discoloration. The color disappeared completely after about 150 s visible irradiation under a flashlight. (c) Reversible photochromism of (±)-7C₂ under UV (λ=365 nm or 254 nm) and visible light. The absorbance was monitored at the absorption peak of the ring-closed DAE at 519 nm.

FIG. 10 is the chemical structure of polymer P1-C₂ and its photochemical property in acetonitrile characterized by UV-vis spectroscopy. (a) Photochromism of the P1-C₂ solution under 365 nm UV irradiation. The PSS was achieved after about 40 s of irradiation. (b) Subsequent visible-light irradiation of this polymer solution resulted in the ring-opening reaction and discoloration. The color disappeared completely after about 150 s of visible irradiation.

FIG. 11 is a graph of UV-vis absorption spectra of an ultrasonicated acetonitrile solution of control polymer P2 (2 mg/mL, 10 min ultrasonication) before and after UV irradiation (λ=365 nm, 1 min). The chain-end control polymer remained photoinert after 10 min ultrasonication.

FIG. 12 is a graph of the GPC characterization of polymer P1σ as a function of ultrasonication time. GPC traces are baseline-corrected by subtracting straight line and then normalized by peak height.

FIG. 13 is a graph of the normalized fluorescence emission spectrum of DAE-containing polymers. Ultrasound-induced stereochemistry conversion of P1s caused a bathochromic shift of its fluorescence by about 15 nm (from the black solid line to the red dash line), which is consistent with the generation of the anti-parallel DAEs stereoisomer (blue dotted line).

### DETAILED DESCRIPTION

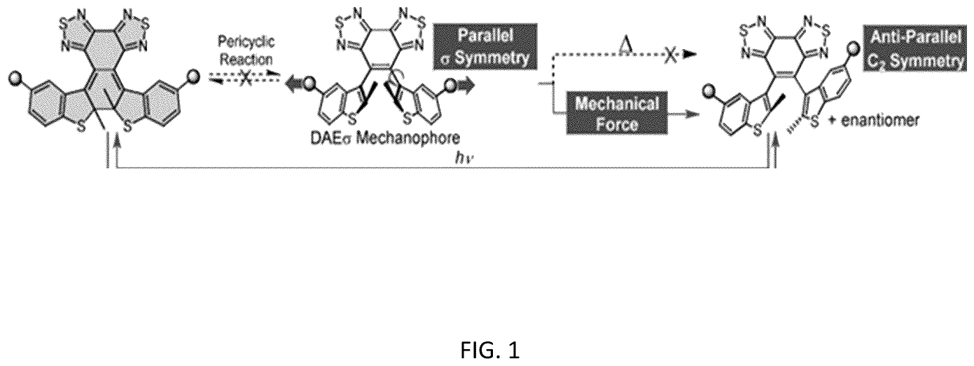

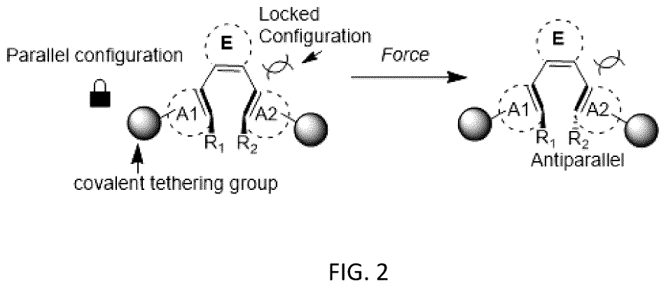

Referring to the figures, wherein like numerals refer to like parts throughout, there is seen in FIG. 1, a sterically congested DAE based mechanophore with mirror symmetry that allows for a new approach to regulate the physical and chemical properties of materials through force-induced stereochemistry conversion. DAEσ is locked in the parallel local conformational minimum and is disallowed to undergo electrocyclization. Mechanical force modifies its rotational potential energy surface and triggers its conversion into the anti-parallel stereoisomer DAE-C2, where the anti-parallel product gains stereoselective activity towards conrotatory 6p photocyclization. Additionally, the mechano-conversion product DAE-C2 shows a change in fluorescence emission in comparison to the parent DAEσ molecule. This proof-of-concept demonstration of force-stereochemistry coupling represents a new, general strategy to manipulate the physical and chemical property of materials using mechanical force and will not only contribute insights into the fundamentally important atropisomerization phenomenon, but also introduces a general strategy for the design of advanced mechanoresponsive materials with stereochemistry-dependent physicological and biological properties that are potentially useful in stress-sensing, medicinal chemistry, information storage, and chiroptic applications. FIG. 2 illustrates the generic design for a mechanophore according to the present invention and FIG. 3 provides several illustrative examples.

The exemplary stereochemistry-converting DAE mechanophore is illustrated as a sterically congested molecular structure where the parallel-DAE mechanophore is incorporated into a polymer backbone through covalent linkage on the benzothiophene so that externally applied mechanical stress is transduced to the DAE motif through the covalently linked polymers. The polymer was connected at the 5-position of the benzothiophene ring in this example of the present invention, although Density Functional Theory Density Functional Theory (DFT) calculations using the constrained geometries simulate external force (CoGEF) technique suggests that the 4-, 6-, and 7-substituted regioisomers are also active toward force-triggered stereochemistry conversion. Elongation of the distance between the anchor points (see FIG. 4) leads to distortion of the angle between the benzothiadiazole bridge and the benzothiophene planes as well as the elongation of bonds along the force transduction axis, and eventually initiates a sudden rotation around the ethene-thiophene single bond and converts the molecular symmetry of the chiral DAE. This rotation across a chiral axis proceeds with an estimated peak force Fₘₐₓ of 0.6 nN (See the SI for details), which is among the lowest Fₘₐₓ values calculated by the CoGEF method across reported mechanophores. Additionally, the strain energy of the constrained DAE drops significantly and approaches zero immediately after the stereochemistry conversion (constrained distance=12.181 Å), as a result of the release of the “hidden length” of 7.337 Å (i.e., a strain of 151% from the constrained distance in the parallel equilibrium geometry).

With the DFT results supporting our hypothesis of force-triggered stereochemistry conversion, the mechanoresponsive chiral materials were synthesized as seen in FIG. 5 to study the force-chirality coupling experimentally. The synthesis of mechanophore 1σ started from treating 5-bromo-2-methyl-1-benzothiophene with n-BuLi followed by reacting the lithiation product with DMF to yield an aldehyde, which was subsequently reduced with NaBH₄ to produce alcohol 2. Alcohol 2 was protected with tert-butyldimethylsilyl chloride to yield compound 3, then brominated with N-bromosuccinimide to obtain the organobromine product 4. 4 was converted to boronic ester 5 by reacting with n-BuLi followed by 2-isopropoxy-4,4,5,5-tetramethyl-1,3,2-di-oxaborolane. Dibromobenzothiadiazole (BBT) was obtained from the bromination of benzothiadiazole, then reacted with boronic ester 5 through a Suzuki-Miyaura cross-coupling reaction to produce the congested DAE 6 as a mixture of near-equivalent parallel and anti-parallel isomers. Atropisomers of 6 do not interconvert and were separated conveniently through silica gel flash chromatography. TBAF deprotection of the desirable parallel atropisomer 66 afforded diol 76, which was esterified with a-bromoisobutyryl bromide to produce the target bi-functional initiator 1a. Living radical polymerization of methyl acrylate with initiator 1a, copper wire, and Me₆ TREN in DMSO afforded poly(methyl acrylate) (PMA) polymer P1σ (Mn=84.4 kDa, D=1.19) containing the stereochemistry-converting mechanophore at near the center of the polymer chain.

The mechanical conversion of the DAEσ mechanophore was studied in solution-phase ultrasonication experiments. Ultrasonication is an effective, and the most widely adopted technique, for studying mechanophore activation in solution. Ultrasound acoustic field causes pressure variations in the solution and generates rapidly collapsing cavitation, inducing a solvodynamic shear force field that transduces force to the backbone of dissolved polymer chains with the force maximized near the chain center. We note that the activation of this type of stereochemistry-converting mechanophores is also possible in solid-state materials such as hydrogels, elastomers, thermosets, and thermoplastic materials. An acetonitrile solution of P1σ (36 mL, 2.0 mg/mL) was subjected to 15 min pulsed U/S (1 s on/2 s off, 0° C., 20 kHz), then the activated polymer was analyzed by NMR spectroscopy. As compared to the 1H NMR spectrum of Pia, the ultrasonicated sample shows a new set of resonances at around 7.69 ppm, 7.08 ppm, and 4.96 ppm that match the structure of a separately synthesized control polymer P1-C2 containing the anti-parallel DAE-C₂, as seen in FIG. 6. NMR results provide direct evidence for the triggered conversion of the chain-centered, parallel DAEσ mechanophore to its anti-parallel stereoisomer DAE-C₂ upon U/S-induced mechanical activation.

As parallel and anti-parallel DAEσ have distinct activities toward pericyclic reaction, 13 we studied the excited-state reactivity of P1σ as a function of ultrasonication time using UV-vis spectroscopy, as seen in FIG. 7. A P1σ solution (12 mL, 2.0 mg/mL in acetonitrile) was subjected to standard ultrasonication conditions. After each duration of ultrasonication, aliquots of the polymer solution were removed and analyzed. The initial polymer P1σ containing the DAE mechanophore was colorless and photoinert. The polymer solutions remained colorless after sonication, as the conversion of DAE stereochemistry was not expected to affect its visible absorption. Notably, UV irradiation (A.=365 nm) turned the ultrasonicated polymer sample into a red color, with an absorption peak emerging at around 520 nm. The photostationary state (PSS) (DAEclosed %=85%) was achieved after about 40 s irradiation using the hand-held UV lamp (see the SI for details). Moreover, the ultrasonicated solution could be switched between the colored and colorless forms reversibly under UV and visible irradiation, respectively. We observed minimal fatigue after five cycles of UV irradiation at 365 nm followed by three cycles at 254 nm. The photochromic property and the absorption profile of the U/S-activated samples match the anti-parallel model materials (±)-7C₂ and P1-C2 (see the SI for details), supporting the hypothesis that U/S activation converts the photoinert parallel DAE into its photoswitchable anti-parallel stereoisomer DAE-C₂. Additionally, the photoswitching property of the U/S-activated materials is persistent over the course of this project, indicating the anti-parallel DAE-C2 product does not thermally atropisomerize to the parent parallel form. The permanent change stereochemistry, molecular symmetry, and chemical activity of our DAE mechanophore is distinct from other noncovalent mechanophores that undergo transient conformational changes under force.

The molecular weights of ultrasonicated polymers were monitored by gel permeation chromatography (GPC). The force-triggered stereoconversion of the DAE mechanophore is a non-covalent process and requires an unusually low magnitude of Fₘₐₓ according to DFT results (vide supra). Indeed, the stereochemistry conversion rapidly approached a plateau after—10 min ultrasonication as demonstrated by the absorption spectra of ultrasonicated samples in PSS as a function of ultrasonication time (FIG. 4b), whereas we only observed a slow decrease in the polymer molecular weight and slight broadening of the polydispersity over the same period (as opposed to scissile mechanophores that result in bimodal distributions upon activation).

To confirm the observed transformation is mechanically triggered, we synthesized a control polymer P2 containing the DAEσ moiety at the end of the polymer chain and subjected it to identical ultrasonication conditions. The control polymer P2 remained photoinert before and after ultrasonication. Since the chain-end DAEσ moiety in the control polymer experienced the same ultrasonication conditions but little force is transduced to the polymer chain end, this control experiment confirms that the observed stereochemistry transformation and the associated chemical property change in our system are resulted from mechanical activation.

In summary, the present invention utilizes a new strategy to manipulate chemical and physical properties of materials through force-stereochemistry coupling. As a proof-of-concept demonstration, a congested parallel DAE molecule with mirror symmetry was converted to its anti-parallel stereoisomer with C2 symmetry under ultrasound-induced force field. Concomitantly, the stereochemistry-converted material is bestowed stereochemistry-selective activity to undergo conrotatory photocyclization reactions between the ring-open and the ring-closed states. The stereochemistry-converted material shows different fluorescence emission in comparison to the inactivated material. This general approach to manipulating molecular stereochemistry, symmetry, and the physicochemical properties of materials contributes new insights into the fundamentally important atropisomerization phenomenon and may lead to the development of advanced mechanoresponsive materials with stereochemistry-dependent physicochemical and biological properties that are potentially useful in stress-sensing, medicinal chemistry, information storage, and chiroptic applications.

What is claimed is:

1. A mechanophore, comprising a molecule having at least one stereocenter that is responsive to an external stimulus to switch from a first stereoisomer into a second stereoisomer of the molecule.

2. The mechanophore of claim 1, wherein the first stereoisomer and the second stereoisomer have different chemical properties.

3. The mechanophore of claim 1, wherein the first stereoisomer and the second stereoisomer have different physical properties.

4. The mechanophore of claim 1, wherein the at least one stereocenter has axial chirality.

5. The mechanophore of claim 1, wherein the first stereoisomer and the second stereoisomer are diastereomers.

6. The mechanophore of claim 1, wherein the molecule includes diarylethene.

7. The mechanophore of claim 1, wherein the molecule comprises sterically congested diarylethene.

8. The mechanophore of claim 1, wherein the molecule comprises a polymer backbone covalently linked to a benzothiophene ring of diarylethene.

9. The mechanophore of claim 8, wherein the molecule has the structure

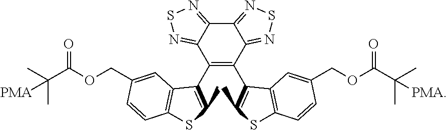

10. A method of responding to an external stimulus, comprising the step of providing a mechanophore formed form a molecule having at least one chiral element that is responsive to the external stimulus to switch from a first stereoisomer into a second stereoisomer of the molecule.

11. The method of claim 10, wherein the first stereoisomer and the second stereoisomer have different chemical properties.

12. The method of claim 10, wherein the first stereoisomer and the second stereoisomer have different physical properties.

13. The method of claim 10, wherein the at least one chiral element has axial chirality.

14. The method of claim 10, wherein the first stereoisomer and the second stereoisomer are diastereomers.

15. The method of claim 10, wherein the molecule includes diarylethene.

16. The method of claim 10, wherein the molecule is sterically congested diarylethene.

17. The method of claim 10, wherein the molecule comprises a polymer backbone covalently linked to a benzothiophene ring of diarylethene.

18. The method of claim 17, wherein the molecule has the structure [chemical structure]
